(19) United States
(12) Patent Application Publication
(10) Pub. No.: US 2020/0218817 A1
(43) Pub. Date: Jul. 9, 2020

(54) SYSTEMS AND METHODS FOR MEDICAL DEVICE AUTHORIZATION

(71) Applicant: Shenzhen Mindray Bio-Medical Electronics Co., Ltd., Shenzhen (CN)

(72) Inventor: James P. Thrower, Oakland, NJ (US)

(21) Appl. No.: 16/241,475

(22) Filed: Jan. 7, 2019

(51) Int. Cl. G06F 21/62; G16H 40/63

(52) U.S. Cl. CPC G06F 21/6218; G06F 3/0488; G16H 40/63

(57) ABSTRACT

A method for medical device authorization includes registering an attempt by a first user to access a user interface of a medical device; detecting a first access device carried by the first user; identifying the first user via the access device; determining whether first user is authorized to access the user interface of the medical device; and in response to the first user being authorized: initiating an access session via the user interface; and logging the first user in connection with the access session.

### TECHNICAL FIELD

The present disclosure relates to medical devices and more particularly to systems and methods for medical device authorization.

### BACKGROUND

Maintaining the security of patient information is an important goal of hospitals and health clinics. Failure to comply with HIPAA (Health Insurance Portability and Accountability Act of 1996) and other privacy regulations with regard to electronic Protected Health Information (ePTI) can result in severe penalties. Well-publicized examples of HIPAA violations involving celebrities make it clear that breaches are frequently perpetrated by employees with at least some legitimate access to patients, hospital information systems, and medical devices containing protected health information. This has necessitated device-level security, including passcodes and the like, for many medical devices.

Unfortunately, each added layer of security introduces pauses into the workflow of healthcare providers. In critical care situations, accumulated delays could negatively impact patient outcomes. What is needed is a way to secure protected health information while minimizing the time required complying with information security procedures.

### BRIEF SUMMARY

According to one aspect of the present disclosure, a method for medical device authorization includes registering an attempt by a first user to access a user interface of a medical device; detecting a first access device carried by the first user; identifying the first user via the access device; determining whether first user is authorized to access the user interface of the medical device; and in response to the first user being authorized, initiating an access session via the user interface and logging the first user in connection with the access session.

Registering the access attempt may include detecting that the user has interacted with the medical device. Alternatively, registering the access attempt may include detecting that the user has broken a light beam between a light source and a photoelectric detector. In other embodiments, registering the access attempt may include detecting that the user has touched the medical device, touched a touch-screen display of the medical device, and/or touched a bezel around a display screen of the medical device. In still other embodiments, registering the access attempt may include detecting the access device.

The method may further include registering an attempt by a second user to access the user interface of the medical device; and in response to not detecting a second access device carried by the second user: terminating the access session; and logging the attempted access by the second user.

The method may further include, in response to detecting that the second user is carrying a second access device, identifying the second user via the second access device; and determining whether the second user is authorized to access the user interface of the medical device.

In one embodiment, the method may further include, in response to the second user being authorized, logging the second user in connection with the access session.

The method may also include, in response to the second user not being authorized, determining whether the second access device is closer to the medical device than the first access device; and if the second access device is closer to the medical device than the first access device, terminating the access session and logging the attempted access by the second user.

In one embodiment, the method may further include terminating the access session after a set time period when the first access device is no longer detected.

In some embodiments, determining whether the first user is authorized may include initiating a limited access session while the first user is being authorized. The limited access session may prevent access of the first user to protected health information. Alternatively, or in addition, the limited access session may prevent access to select functions of the medical device. In one embodiment, identifying the first user may include initiating a limited access session while the first user is being identified.

In one embodiment, the access device includes a radio frequency device, and determining comprises detecting a signal produced by the radio frequency device. In one embodiment, the first access device stores an identification code, and identifying the first user comprises identifying the first user based on the identification code.

In one embodiment, the method may further include disassociating the identification code from the first user after a set period of time. The method may also include wirelessly updating the identification code stored by the first access device.

In another aspect, a system for medical device authorization may include a sensor to register an attempt by a first user to access a user interface of a medical device; a wireless receiver to detect a first access device carried by the first user; and a processor, in communication with the sensor and wireless receiver, to identify the first user via the access device; determine whether first user is authorized to access the user interface of the medical device; and, in response to the first user being authorized, initiate an access session via the user interface and log the first user in connection with the access session.

### BRIEF DESCRIPTION OF THE DRAWINGS

The present disclosure may best be understood by reference to the following description taken in conjunction with the accompanying figures, in which like parts are referred to by like numerals.

FIG. 1 illustrates a conventional technique for medical device authorization;

FIG. 2 illustrates a technique for medical device authorization according to one embodiment of the present disclosure;

FIG. 3 illustrates various methods for registering an access attempt;

FIG. 4 illustrates a process of terminating an access session;

FIG. 5 illustrates a process of logging a second user in connection with an access session;

FIG. 6 illustrates a technique for updating an access device;

FIG. 7 is a schematic block diagram showing details of a medical device and an access device; and

FIGS. 8, 9, and 10 are flowcharts of methods for medical device authentication.

### DETAILED DESCRIPTION

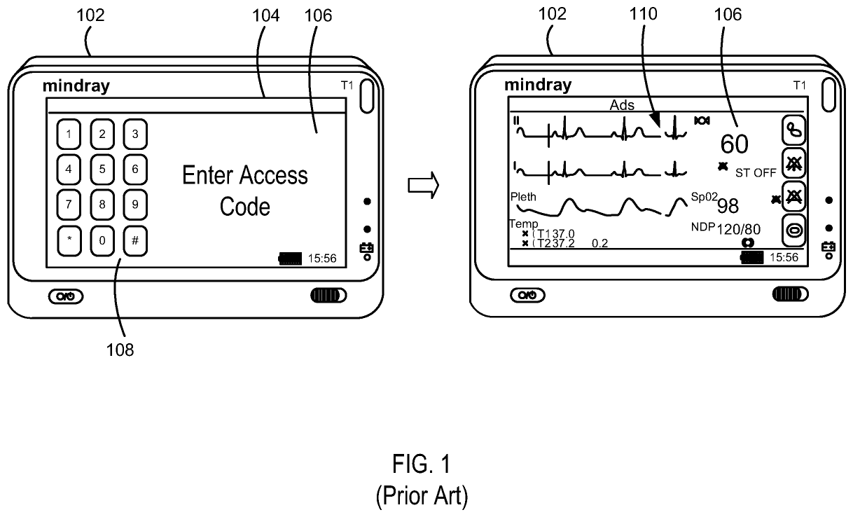

The present disclosure is directed to systems and methods for systems and methods for medical device authorization. FIG. 1 illustrates a conventional medical device 102, such as a patient monitor, ultrasound imaging device, blood analyzer, or the like. The medical device 102 includes a display screen 104 upon which is displayed a graphical user interface (GUI) 106. To prevent unauthorized access to the medical device 102 and possible breaches of patient confidentiality, the GUI 106 may provide a virtual keypad 108, which a user, such as a doctor or nurse, may employ to enter an access code. While a virtual keypad 108 is illustrated, those skilled in the art know that conventional medical devices 102 may variously include physical (or virtual) keys, buttons, dials, sliders, trackballs, mice, or other controls for interacting with the medical device 108.

In response to entering a valid access code using the virtual keypad 108, the medical device 102 may be unlocked, displaying a GUI 106 for controlling and/or receiving information from medical device 110. For example, the GUI 106 may include a series of waveforms 110 representing patient parameters, such as EEG, heart rate, temperature, etc., received over time from one or more sensors (not shown) coupled to the patient. An unlocked GUI is referred to herein as an “access session,” which may be variously referred to in the art as a “login session.” In computing, a login session is the period of activity between a user logging in and logging out of a system.

Unfortunately, each medical device 102 will generally have a different access code, requiring the user to remember multiple codes for different device types and possibly different devices of the same type. In some cases, the user will have a personal (user-specific) access code for a particular medical device that is rarely applicable to another medical device.

Aside from the inconvenience of having to remember multiple access codes, every pause or delay in the workflow of a medical professional can introduce the risk of negative patient outcomes. In critical care situations, the combined delays introduced by authorizing a user to access to different medical devices 102 can be unacceptably large, putting patients' lives at risk.

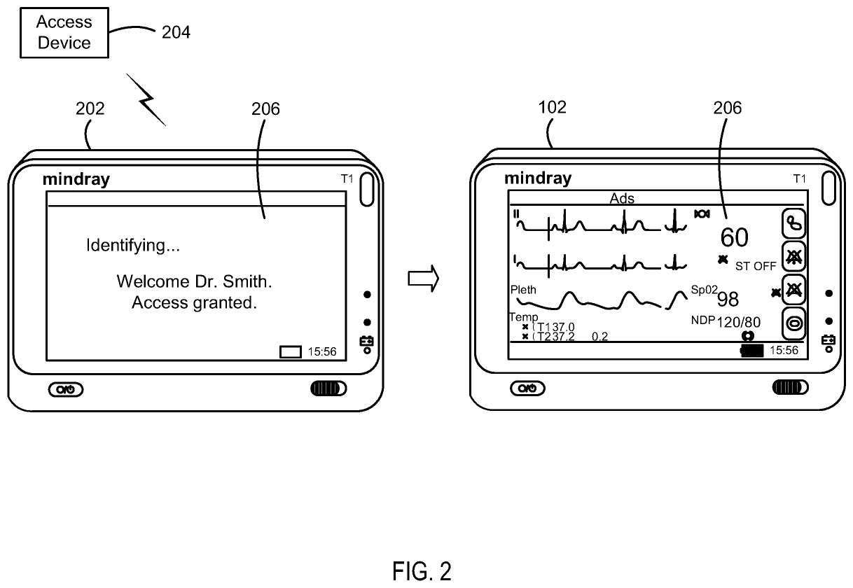

FIG. 2 illustrates a medical device 202 in accordance with the present disclosure that solves the aforementioned problems. In one embodiment, each user is provided with an access device 204, which may be worn or carried by the user. The access device 204 may be embodied as an access card carried in a user's wallet or a lanyard around the user's neck. The access device 204 may also be integrated into a pin, watch, clothing, cell phone, or other object commonly carried or worn by the user.

The access device 204 may employ various wireless communication technologies, such as radio frequency identification (RFID), near-field communication (NFC), Bluetooth, or W-Fi (802.11), to wirelessly communicate with a suitably equipped medical device 202. Those skilled in the art will recognize that other wireless communication technologies may be used within the scope of the present disclosure.

As described more fully below, in response to the user attempting to access a user interface of the medical device 202, the access device 204 carried by the user may be detected, and the user may be identified and authorized via the access device 204. In response to the user being authorized, an access session on the medical device 202 may be initiated. For example, a GUI 206 may welcome the user and indicate that access is granted, after which the GUI 206 may switch to a waveform display on a patient monitor, as shown in FIG. 2, providing full user access.

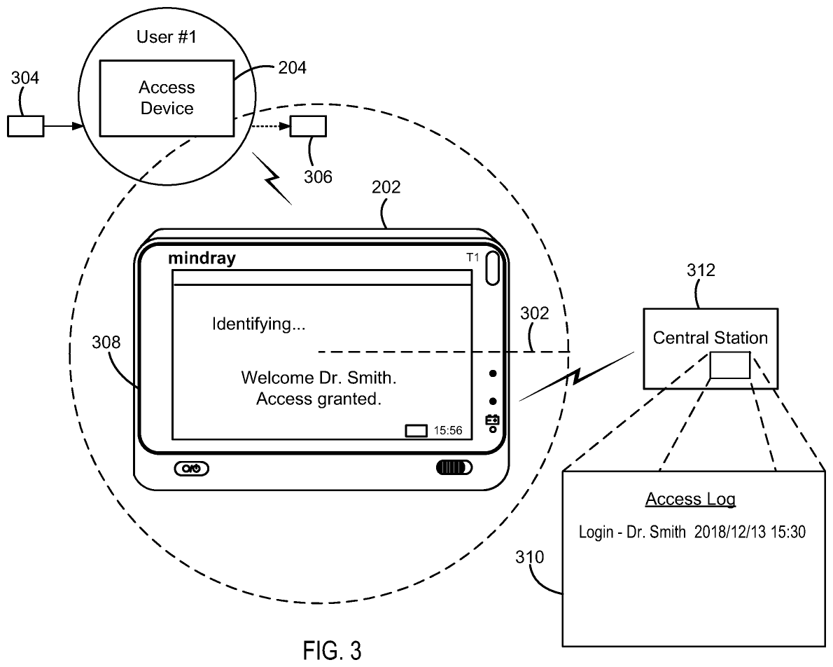

Referring to FIG. 3, a user's attempt to access the user interface of the medical device 202 (triggering authentication via the access device 204) may be registered in various ways. In one embodiment, registering the access attempt may include detecting that the user has interacted with the medical device 202 via a control mechanism or in other ways. This may include touching the medical device, touching a touch-screen interface of the medical device, and/or attempting to control the device using a predetermined gesture, vocalization, or the like. In other embodiments, the medical device may include a touch-sensitive region, such as a touch pad, touch ring, or the like. For example, the touch ring may extend around a bezel 308 of the medical device's display screen.

In another embodiment, the user carrying the access device 204 may break a light beam being sent between a light source 304 and a photoelectric detector 306. The light source 304 and photodetector 306 may be in wired or wireless communication with the medical device 202.

In yet another embodiment, the access device 204 may be brought within a particular radius (distance) 302 of the medical device 302, which may include the distance at which the access device 204 may be detected by the medical device 202 based on the wireless technology employed. In this case, the “attempt” by the user may not reflect the user's subjective intent, but rather the system's interpretation of the user's intent based on his or her proximity to the medical device 202. Thus, registering a user's “attempt” should not be limited only to situations in which the user physically interacts with the medical device 202. In some cases, the interaction may be that the user's face was recognized by the medical device 202.

The access or attempted access may be logged in an access log 310 within a central station 312, such as a central nursing station, hospital server, or the like. Alternatively, or in addition, the access log 310 may also be stored in the medical device 202. The access log 310 may identify the user carrying the access device 204, the user's login and logout times, whether an access attempt was authorized, etc.

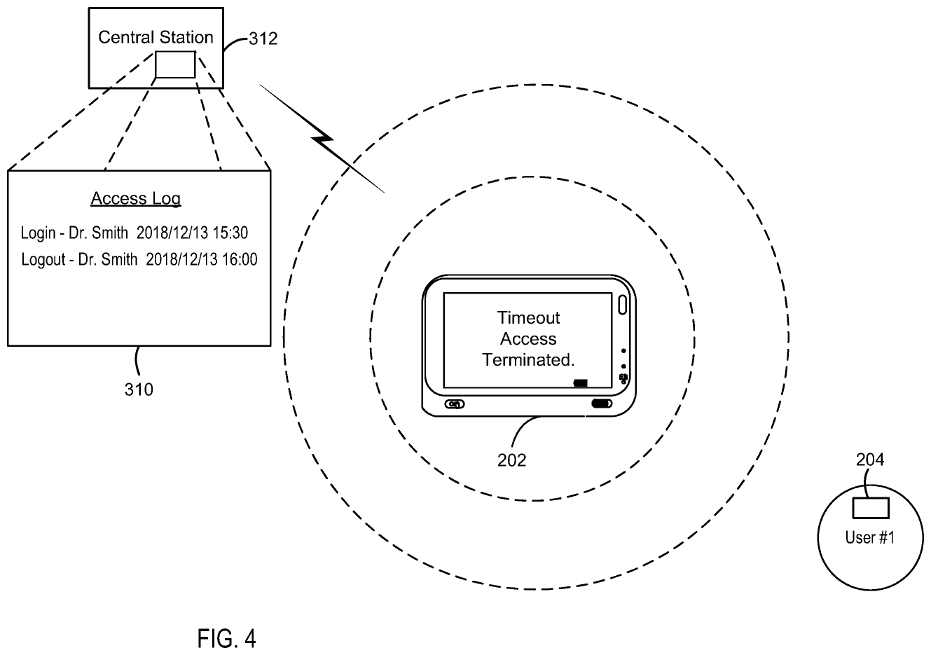

Referring to FIG. 4, once an access session has been established, it may be terminated under certain conditions without the user explicitly logging out of the system. In one embodiment, an access session may terminate after a certain amount of time has elapsed if the user carries the access device 204 outside of a particular radius of the medical device 202. The radius may be a particular distance determined, for example, by wireless signal strength, GPS location, or the like. Alternatively, the radius may be the distance at which the access device 204 can no longer be detected by the medical device 202, which will vary based on the technology employed. If the user leaves the radius, a timeout or countdown may be initiated. At the end of the timeout, if the user still remains outside the defined radius, the access session may be automatically terminated. However, in one embodiment, if the user returns to within the radius before the timeout expires, the timeout may be halted and/or reset.

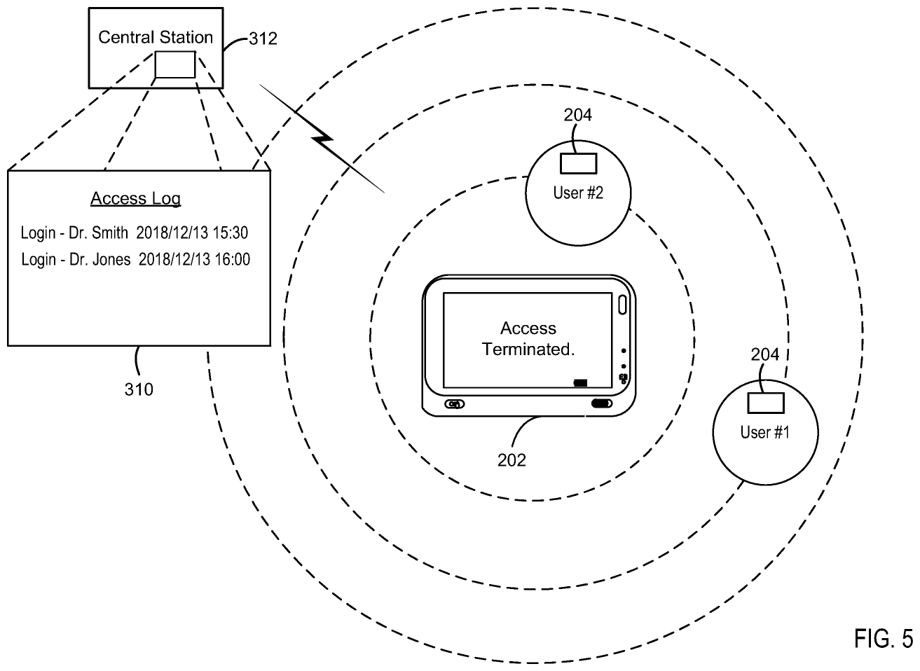

Referring to FIG. 5, a second user may attempt to access the user interface of the medical device 202 after the first user has established an access session but has not yet logged out (and the access session has not been automatically terminated). For example, the first user may temporarily leave the immediate proximity of the medical device 202, after which another doctor or nurse may wish to use the medical device 202. How the medical device 202 responds may depend on whether the second user is carrying an access device 204, whether the access device identifies the user as someone with authorization to use the medical device 202, and/or the relative distances of the first and second user to the medical device 202.

For example, if the second user attempts to access the medical device 202, such as by touching the display screen, the medical device 202 may detect whether the user is carrying an access device 204. If not, the access session may be immediately terminated and an indication of the access attempt stored in the access log 310. In some situations, an alarm may be optionally initiated at the medical device 202 and/or central station 312.

If the user does have an access device 204, the user may be identified via the access device 204 and a determination made whether the user is authorized to use the medical device 202. If the user is not authorized, the access session may be immediately terminated, the access attempt logged, and/or an alarm initiated. On the other hand, if the user is authorized, the access by the second user may be logged in the access log, as depicted in FIG. 5.

In another embodiment, if the user is found to be not authorized, whether the access session is terminated may be based on the relative distances of the first and second users to the medical device 202. For example, if the first user is closer to the medical device than the second user, the presence of the second user may be simply logged in the access log 310. Alternatively, if the second user is closer than the first user, the access session may also be terminated.

In some cases, either the first or second user may be provided with temporary, limited access while the user is being identified and/or authorized. For example, the limited access session may prevent access to protected health information and/or certain functions of the medical device, such as defibrillation. The limited access system may last while the user is being identified and/or authorized or it may terminate after a particular amount of time. During a limited access session, all actions by the first user may be logged in the identity database to permit subsequent review by authorized personnel.

Even in cases where a user does not have an access device 204 or the user fails authorization, there may be situations in which providing a limited access session may be desirable, such as in an emergency. The limited access session may be initiated automatically upon failing to detect an access device 204 or the user failing authorization. Alternatively, the limited access session may be manually initiated by entering a code on a virtual keyboard 108, as shown in FIG. 1.

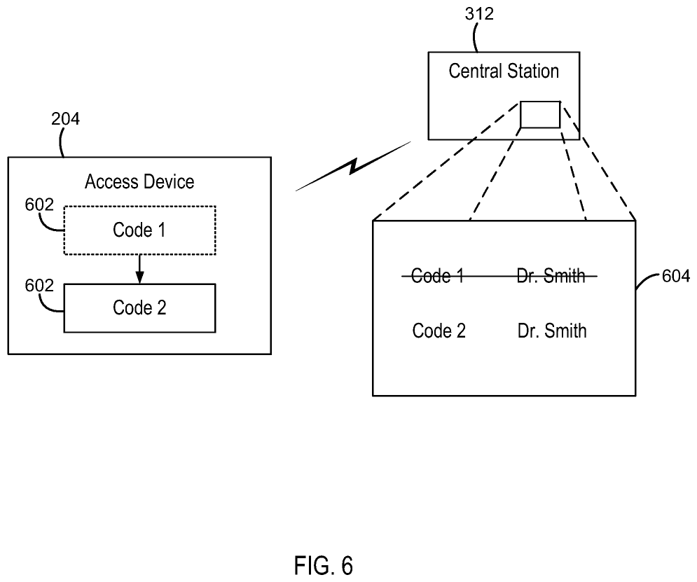

Referring to FIG. 6, each access device 204 may store at least one code 602 in a read-only or programmable memory device. A corresponding code may be stored in an identity database 604 within the central station 312 and/or the medical device 202. The identity database 604 may relate codes 602 to specific users or classes of users.

In one embodiment, the codes 602 stored in the identity database 604 may be specifically or periodically disassociated from a user. This may occur, for example, when a user's employment is terminated. Alternatively, the code may be periodically invalidated and changed for security purposes. In such a case, the current code 602 in the access device 204 may no longer be valid to access the medical device 202. In the case of a read-only code 602, the user may be required to return the access device 204 to the hospital's HR department and receive a new access device 204.

In other embodiments, the code 602 in the access device 204 may be wirelessly updated. For example, as shown in FIG. 6, “code 1” may be invalidated and a new “code 2” associated with Dr. Smith. In such a case, the central station 312 may send a command to the access device 204 to replace “code 1” with “code 2.” This may occur, for example, when the user brings the access device 204 in proximity to the medical device 202 or at other designated locations within a hospital.

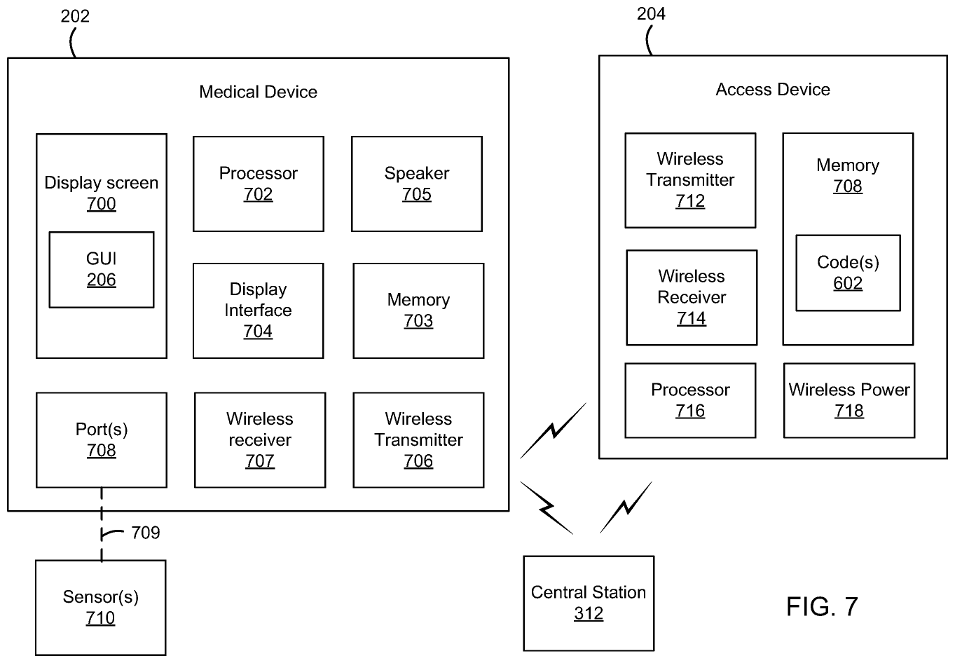

FIG. 7 is a schematic diagram of an exemplary hardware environment for a system for implementing the operations described in FIGS. 2-6. In the depicted embodiment, the medical device 202 is a patient monitor. However, in other embodiments, the medical device 202 could be ultrasound imaging system, a blood analyzer, an anesthesia machine, or the like. Thus, some components described herein that are specific to a patient monitor need not be included in every embodiment.

The medical device 202 may include a display screen 700, a processor 702, a memory 703, a display interface 704, a speaker 705, a wireless transmitter 706, a wireless receiver 707, and one or more ports 708, which may be electrically (via wire leads 709) or wirelessly coupled to one or more sensors 710 that read a patient's vital signs.

The display screen 700 may be embodied as a liquid crystal display (LCD), a light-emitting diode (LED) display, an organic light-emitting diode (OLED) display, or other similar display device, and may be touch-sensitive. The processor 702 may be a general purpose microprocessor, an application specific processor (ASP), a digital signal processor (DSP), or the like. The memory 703 may include volatile and non-volatile memory using any combination of random access memory (RAM), read-only memory (ROM), electrically erasable programmable read-only memory (EEPROM), magnetic storage, optical storage, or the like.

The display interface 704 may be implemented using general purpose or custom graphics hardware capable of generating a digital or analog signal including the GUIs 206 shown in FIG. 2. The port(s) 708 may be implemented using standard connectors for coupling to the sensor(s) 710. In some embodiments, the port(s) 708 may use the Universal Serial Bus (USB) 3.0 standard, although other technologies may be used. As used herein, “port” may refer to not only a connector, but any or all circuitry used to convert signals received from the sensors 710 into digital data that can be manipulated by the processor 702. As such, a port 708 may include an analog-to-digital converter (ADC), one or more filters, etc., where a sensor 710 outputs analog signals. Likewise, a port 708 may include hardware for wirelessly receiving parameter data from a suitably equipped sensor 710.

The transmitter 706 and receiver 707 may operate in very-high frequency (VHF) or ultra-high frequency (UHF) wireless bands. In the United States, the wireless medical telemetry service (WMTS) provides dedicated protected bands which have been allocated for this purpose and which many hospitals prefer to use over the more widely used industrial, scientific, and medical (ISM) radio bands. Currently the WMTS provides licensed bands in a 608 to 614 megahertz (MHz) range (also known as the 608 MHz band), a 1395 to 1400 MHz range (also known as the 1400 MHz band), and a 1427 to 1432 MHz range. The ISM bands include the popular 2.4 to 2.5 gigahertz (GHz) range and a 5.725 to 5.875 GHz range (also known as the 2.5 GHz and 5 GHz bands) which may be used by routers, wireless home telephones, or the like. Hospitals and other medical providers often use the WMTS bands over the ISM bands because they require less active management and present a smaller patient safety risk.

Some medical devices 202 may use the same protected WMTS bands but have bidirectional radios which allow the device to have additional functionality because they are able to receive, as well as send, data. In other embodiments, the transmitter 706 and/or receiver 707 may implement the IEEE 802.11 standard (known to industry groups as Wi-Fi), or other wireless protocols. In some embodiments, the wireless transmitter 706 and wireless receiver 707 may be embodied in a single transceiver component.

An access device 204 may include a wireless transmitter 712 and optionally a wireless receiver 714 (to permit, for example, remote updating as discussed with reference to FIG. 6). The wireless transmitter 712 and wireless receiver 714 may implement wireless protocols compatible with the wireless transmitter 706 and wireless receiver 707 of the medical device 202, as well as wireless components (not shown) in the central station 312. Of course, the wireless transmitter 712 and wireless receiver 714 may implement different protocols and/or be combined in various embodiments.

The access device 204 may also include a processor 716 and a memory 708, which may be used to store the code(s) 602 discussed in FIG. 6. In one embodiment, the access device 204 may include a wireless power adapter 718 that may power the access device 204 when it is brought into range of a suitable power source that generates a time-varying electromagnetic field. For longer distance transmission, the wireless power adapter 718 may implement COTA™ technology, in which a power transmitter uses a 2.4 GHz channel and array of directional antennas to triangulate a power receiver.

The access device 204 may include other components, such as a global positioning system (GPS) device (not shown) for determining the position of the access device 204, e.g., relative to the medical device 202. As GPS is unreliable indoors, the access device 204 may, in combination, use the wireless receiver 714 to determine its location based on known locations of wireless transmission sources within a building by triangulation or other known techniques.

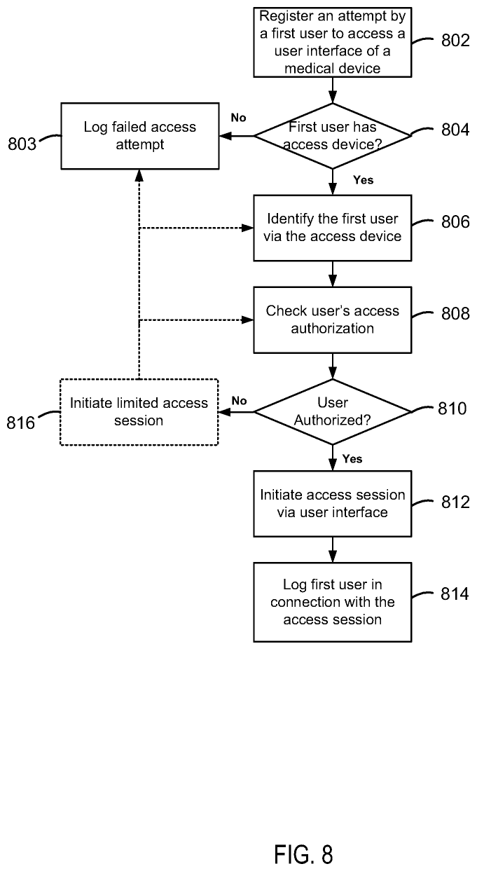

FIG. 8 is a flowchart of a method for one embodiment of a method for medical device authorization. The described operations may be performed by the hardware environment shown in FIG. 7 or using any suitable combination of hardware, software, and firmware. Various steps may be performed medical device 202, the access device 204, and/or the central station 312, referred to hereafter as the “system.” A skilled practitioner will recognize that the system may include other components not specifically interested but known in the art.

The central station 312 may likewise be implemented using general or special-purpose computing hardware and include components similar to those discussed in connection with the medical device 202, the details of which will not be repeated here.

FIG. 8 is a flowchart of a method for medical device authorization according to one embodiment of the present disclosure. Initially, a system (which could include the system depicted in FIG. 7) registers 802 an attempt by a first user to access a user interface of a medical device. For example, the user may touch the medical device, approach the medical device, and/or make a particular gesture or vocalization recognized by the system as a command. A determination 804 is made whether the first user has an access device, as described above, by detecting electromagnetic waves emitted by the access device. If the first user does not have an access device, the system may log a failed access attempt in an access log within the medical device and/or the central station. In some cases, an alarm may be generated.

If, however, the user has an access device, the system identifies 806 the first user via the access device. This may include reading a code from the access device and looking up the code in an identity database. In some cases, biometric identification, such as facial recognition or the like, may also be employed. As used herein, identification does not necessarily mean determining the name of the user. Rather, the system may determine that the user is among a class of authorized users by virtue of a code stored within the access device.

The user's identity (name or identifying code) is checked 808 for authorization to access the medical device in question. In some cases, this step may be performed at the same time that the user is identified. If the user is authorized to use the medical device, the system may initiate 812 an access session via the user interface in which the user may control and/or receive information from the medical device. In addition, the first user may be logged 814 in connection with the access session in an access log or the like within the medical device and/or the central station.

If, however, the first user is not authorized to use the medical device, the system may log 803 the failed access attempt as discussed above. Alternatively, or in addition, the system may initiate 816 a limited access session for the first user. Occasionally, the authentication process, particularly if done wirelessly via a remote identity database, may not be immediate, such as when a network connection is not available. In other situations, it may be necessary for a user to access a medical device in an emergency. In such cases, there may be a need to provide temporary, limited access while the user is being identified and/or authorized. As noted above, such access may carry various restrictions. For example, the limited access session may prevent access to historical patient data and/or certain functions of the medical device. The limited access system may last while the user is being identified and/or authorized or it may terminate after a particular amount of time. During a limited access session, all actions by the first user may be logged in the access log to permit subsequent review by authorized personnel.

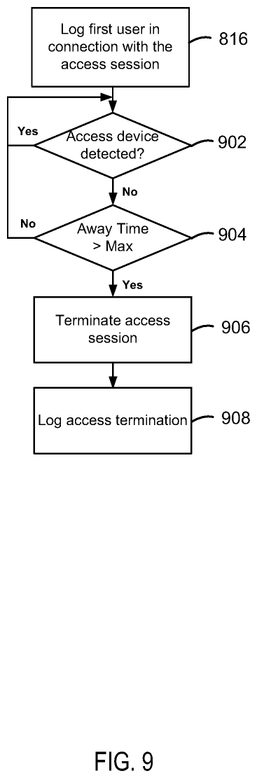

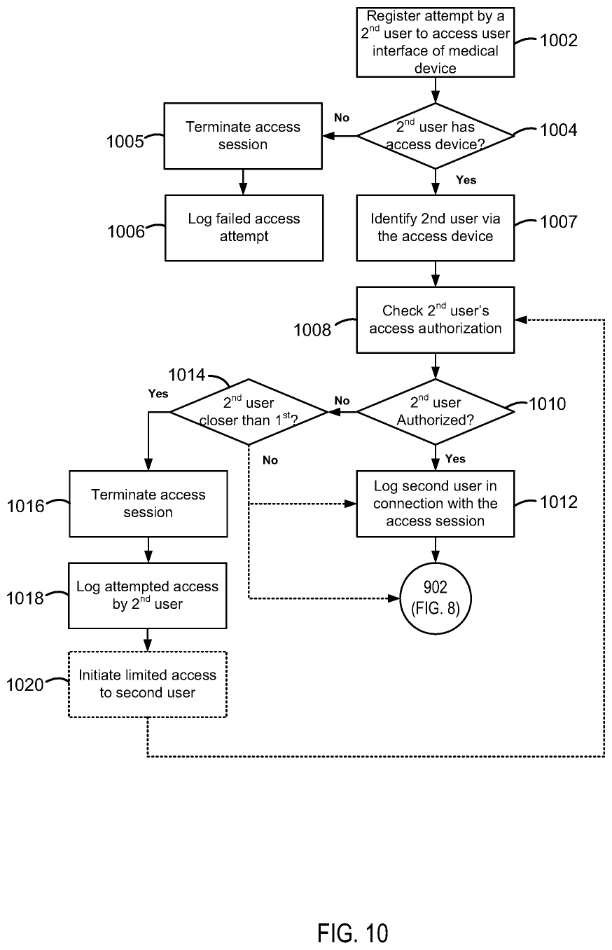

Referring to FIG. 9, the flowchart begins with step 816 of the flowchart of FIG. 8, where the first user is logged in connection with the access session and is able to use the medical device. In some cases, the first user may depart from the immediate vicinity of the medical device to perform other tasks, such as manually taking vital signs, connecting sensors, responding to a user request, etc. Whether the access session remains active may depend on the distance of the user from the medical device, the amount of time the user is away from the medical device, and/or whether a second person enters the room and/or attempts to access the medical device (as will be discussed in more detail in conjunction with FIG. 10).

In one embodiment, a determination 902 is made whether an access device is detected or within a particular distance/radius from the medical device. Every wireless technology has a limited range. Therefore, the radius could be defined as the maximum distance at which the access device can be detected. For example, a class three radio for a Bluetooth device may have a range of up to 1 meter, whereas a class 2 radio has a range of 10 meters.

If the access device of the first user is detected, the process may loop indefinitely until the access device is no longer detected. Alternatively, the process may loop for a set period of time after which the access session may terminate regardless of whether the access device is detected.

If the access device is not detected, a determination 904 may be made whether the amount of time the access device has not been detected (referred to herein as “Away Time”) is greater than a maximum value. If the Away Time exceeds the maximum value, the access session is terminated 906 in one embodiment, and the termination is logged 908.

If the Away Time is less than or equal to the maximum value, control may again pass to step 902, where a determination is made whether the access device is detected. Although not specifically illustrated, it may be implied by the definition of Away Time that the Away Time may be reset to zero if the user returns and his or her access device is again detected.

FIG. 10 is a flowchart of a process for handling the introduction of a second user to the methods discussed in FIGS. 8 and 9. It is understood that a first user has already been authorized and an access session initiated. Initially, the system registers 1002 an attempt by a second user to access the user interface of the medical device. The access attempt may be similar to that described above for the first user. For example, the second user could touch or approach the medical device. A determination 1004 may be made whether the second user has an access device. If not, the system may terminate 1005 the access session and log 1006 the failed access attempt.

If, however, the second user has an access device, the system identifies 1007 the second user via the access device and checks 1008 whether the second user is authorized to user the access device. In one embodiment, if the second user is authorized, the system may log 1012 the second user in connection with the access session (as illustrated in FIG. 5), after which control returns to step 902 of FIG. 8.

If the second user is holding an access card but is not authorized, the system may respond in various ways. For example, as illustrated, the system may determine 1014 whether the second user is closer to the medical device than the first user. This may occur, for example, when the first user has temporarily walked away from the medical device without logging out. In such a case, the first user may not be detected, or may be detected at a greater distance from the medical device than the second user, such as by comparing the respective signal strengths from the access devices of the first and second users.

In one embodiment, if the second user is closer to the medical device than the first user, the system may terminate 1016 the access session, log 1018 the attempted access by the second user and optionally initiate 1020 a limited access session for the second user, as described earlier. If, however, the second user is not closer to the medical device than the first user, in one embodiment, the presence of the second user may be logged 1012 in connection with the access session. Alternatively, the second user is not logged and control passes to step 902 of FIG. 8.

In one embodiment, either the first or second user, once authorized, may be given an option for selecting one or more users responsible for the access session. This may determine, for example, which user(s) must remain in proximity to the medical device to keep the access session active, which user(s) departure or arrival should be noted in the access log, etc.

This disclosure has been made with reference to various exemplary embodiments including the best mode. However, those skilled in the art will recognize that changes and modifications may be made to the exemplary embodiments without departing from the scope of the present disclosure. For example, various operational steps, as well as components for carrying out operational steps, may be implemented in alternate ways depending upon the particular application or in consideration of any number of cost functions associated with the operation of the system, e.g., one or more of the steps may be deleted, modified, or combined with other steps.

Additionally, as will be appreciated by one of ordinary skill in the art, principles of the present disclosure may be reflected in a computer program product on a computer-readable storage medium having computer-readable program code embodied in the storage medium. Any tangible, non-transitory computer-readable storage medium may be utilized, including magnetic storage devices (hard disks, floppy disks, and the like), optical storage devices (CD-ROMs, DVDs, Blu-Ray discs, and the like), flash memory, and/or the like. These computer program instructions may be loaded onto a general purpose computer, special purpose computer, or other programmable data processing apparatus to produce a machine, such that the instructions that execute on the computer or other programmable data processing apparatus create means for implementing the functions specified. These computer program instructions may also be stored in a computer-readable memory that can direct a computer or other programmable data processing apparatus to function in a particular manner, such that the instructions stored in the computer-readable memory produce an article of manufacture, including implementing means that implement the function specified. The computer program instructions may also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

While the principles of this disclosure have been shown in various embodiments, many modifications of structure, arrangements, proportions, elements, materials, and components, which are particularly adapted for a specific environment and operating requirements, may be used without departing from the principles and scope of this disclosure. These and other changes or modifications are intended to be included within the scope of the present disclosure.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. As used herein, the terms “comprises,” “comprising,” and any other variation thereof, are intended to cover a non-exclusive inclusion, such that a process, a method, an article, or an apparatus that comprises a list of elements does not include only those elements but may include other elements not expressly listed or inherent to such process, method, system, article, or apparatus. Also, as used herein, the terms “coupled,” “coupling,” and any other variation thereof are intended to cover a physical connection, an electrical connection, a magnetic connection, an optical connection, a communicative connection, a functional connection, and/or any other connection.

Those having skill in the art will appreciate that many changes may be made to the details of the above-described embodiments without departing from the underlying principles of the invention. The scope of the present invention should, therefore, be determined only by the following claims.

What is claimed is:

1. A method comprising:
registering an attempt by a first user to access a user interface of a medical device;
detecting a first access device carried by the first user;
identifying the first user via the access device;
determining whether first user is authorized to access the user interface of the medical device; and
in response to the first user being authorized:
initiating an access session via the user interface; and
logging the first user in connection with the access session.

2. The method of claim 1, wherein registering the access attempt comprises detecting that the user has interacted with the medical device.

3. The method of claim 1, wherein registering the access attempt comprises detecting that the user has broken a light beam between a light source and a photoelectric detector.

4. The method of claim 1, wherein registering the access attempt comprises detecting that the user has touched the medical device.

5. The method of claim 4, wherein registering the access attempt comprises detecting that the user has touched a touch-screen display of the medical device.

6. The method of claim 4, wherein registering the access attempt comprises detecting that the user has touched a bezel around a display screen of the medical device.

7. The method of claim 1, wherein registering the access attempt comprises detecting the access device.

8. The method of claim 1, further comprising:
registering an attempt by a second user to access the user interface of the medical device; and
in response to not detecting a second access device carried by the second user:
terminating the access session; and
logging the attempted access by the second user.

9. The method of claim 8, further comprising:
in response to detecting that the second user is carrying a second access device:
identifying the second user via the second access device; and
determining whether the second user is authorized to access the user interface of the medical device.

10. The method of claim 9, further comprising:
in response to the second user being authorized, logging the second user in connection with the access session.

11. The method of claim 9, further comprising:
in response to the second user not being authorized:
determining whether the second access device is closer to the medical device than the first access device; and
if the second access device is closer to the medical device than the first access device:
terminating the access session; and
logging the attempted access by the second user.

12. The method of claim 1, further comprising:
terminating the access session after a set time period when the first access device is no longer detected.

13. The method of claim 1, wherein determining comprises:
initiating a limited access session while the first user is being authorized.

14. The method of claim 13, wherein the limited access session prevents access of the first user to historical patient data.

15. The method of claim 13, wherein the limited access session prevents access to select functions of the medical device.

16. The method of claim 1, wherein identifying comprises:
initiating a limited access session while the first user is being identified.

17. The method of claim 1, wherein the access device comprises radio frequency device, and wherein detecting comprises detecting a signal produced by the radio frequency device.

18. The method of claim 1, wherein the first access device stores an identification code, and wherein identifying the first user comprises identifying the first user based on the identification code.

19. The method of claim 18, further comprising:
disassociating the identification code from the first user after a set period of time.

20. The method of claim 18, further comprising:
wirelessly updating the identification code stored by the first access device.

21. A system comprising:
a sensor to register an attempt by a first user to access a user interface of a medical device;
a wireless receiver to detect a first access device carried by the first user;
a processor, in communication with the sensor and wireless receiver, to:
identify the first user via the access device;
determine whether first user is authorized to access the user interface of the medical device; and
in response to the first user being authorized:
initiate an access session via the user interface; and
log the first user in connection with the access session.

22-40. (canceled)
